(19) United States
(12) Patent Application Publication
(10) Pub. No.: US 2009/0313865 A1
(43) Pub. Date: Dec. 24, 2009

(54) Scent delivery system for greeting cards, envelopes and bags

(76) Inventors: Stephen Vincent Peretti, Wellesley Hills, MA (US); Rita Marlene Brett-Bold, Glendale, CA (US)

Correspondence Address: ROBERT NATHANS, 36 STAG DRIVE, BILLERICA, MA, 01821, US

(21) Appl. No.: 12/214,589

(22) Filed: Jun. 20, 2008

(51) Int. Cl. B42D 15/02; A61L 9/04

(52) U.S. Cl. 40/124.11; 283/117; 239/60

(57) ABSTRACT

A scent strip is applied to envelopes and bags as well as greeting cards sold in stores and includes a plastic foam elastomeric substrate containing micro-encapsulated scented material. A marketing activation tab is affixed to a protective release liner to aid in manually peeling the liner off of the greeting card while at the same time preventing the greeting card from being inserted into an envelope used to send the greeting card, unless the protective security release liner is separated from the greeting card, enabling subsequent scent release by a recipient of the card. Also, the protective security release liner has a small window therein, enabling a prospective product purchaser to scratch the scent strip and sniff a small amount of sampled scented material passing through the small window from the scent material containing substrate, and yet preventing undesirable release of substantial scent material within a store selling the product.

### BACKGROUND OF THE INVENTION

The emerging scent delivery market is enabling consumers to enjoy refined scents that provoke memories and add pleasant experiences to environments and products.

U.S. Pat. No. 6,686,013 issued Feb. 3, 2004 to Tebbe, discloses in FIGS. 3 and 4 a folding greeting card which has a micro-capsule layer, the micro-capsules of which contain a perfume and can be destroyed mechanically. The micro-capsule layer 48 is firmly joined to one half of the greeting card via an adhesive binder layer 44 and is joined to the other half of the card by a self-adhesive layer 46 when the greeting card is closed. When the card is opened the micro-capsules are destroyed and the perfume they contain is released.

A lower protective layer 52, which is shown in dashed lines only in FIG. 3 was removed when the perfume pad 36 was bonded into the double card 10, and an upper protective layer 54 which is still to be removed by the sender of the greeting card, further belongs to the perfume pad 36. Upon removal of the upper protective layer 54, the card is closed and sent to the recipient who opens the card to release the perfume when the micro-capsules are destroyed.

The Tebbe disclosure states: The invention was explained above with reference to various greeting cards. It will be understood that it may also be used in particular in the field of packs. Packs are often folded from flat sections of material and thus necessarily have superimposed material portions which are joined by adhesive. The invention may be used at all such joining points in order to release a perfume when the joining point is opened. One need only think of envelopes, packs for foodstuffs etc.

The Tebbe disclosure goes on to state: that an intermediate product can be provided comprises a first self-adhesive layer and a second self-adhesive layer as well as a capsule layer which comprises micro-capsules which contain at least one perfume and can be mechanically destroyed, and lies between the two self-adhesive layers and the capsule layer lying between them. A development of the intermediate product facilitates handling by an end user. A development of the invention makes it possible to use the intermediate product like a transparent packing strip. Particularly when packing gifts, the gift wrap used thus remains fully visible, so that the bonding point is not noticeable.

An intermediate product is also already transparent when it is joined to an article while not yet activated, i.e. carries a further protective layer. This is desirable for articles produced in small quantities using intermediate products.

### BRIEF SUMMARY OF PREFERRED EMBODIMENTS OF THE INVENTION

The present invention has various features that are improvements over the embodiments of the aforesaid Tebbe patent.

The scent strip of the invention (FIG. 1), that can be applied to envelopes and bags as well as greeting cards, has a plastic foam substrate including micro-encapsulated scented material that provides various benefits including cushioning the micro-encapsulated scented material during handling, thereby to deter undesired scent release.

The elastomeric substrate has a pattern of cavities therein containing micro-encapsulated scented material, the cavities being separated from one another by a connecting layer of micro-encapsulated slurry substantially thinner than heights of the scented cavities, causing audible and tactile effects upon final activation of the scent strip to deploy the scent by the recipient of the product bearing the scent strip.

A marketing activation tab is affixed to a protective security release liner to aid in manually peeling the security release liner off of the greeting card scent strip while at the same time preventing the greeting card from being inserted into an envelope used to send the greeting card, unless the protective security release liner is separated from the greeting card, such separation enabling subsequent scent release by a recipient of the greeting card upon her removal of an overlying scent activation panel from the scent strip.

In accordance with another feature of the invention, the protective security release liner has a small window therein, enabling a prospective greeting card or other product purchaser to scratch the scent strip and sniff a small amount of sampled scented material passing through the small window from the scent material containing substrate, and yet preventing undesirable and wasteful release of substantial scent material within a store carrying the greeting cards.

### BRIEF DESCRIPTION OF THE DRAWINGS

Other features and advantages of the invention will become more apparent upon study of the following description taken in conjunction with the drawings in which:

FIG. 1 is a cross-section of the scent delivery device including the aforesaid scent strip;

FIG. 2 is a perspective view of a greeting card viewed from the inside of the card;

FIG. 3 is a perspective view of a greeting card viewed from the outside of the card;

FIG. 4 shows components of the delivery device applied to an envelope; and

FIG. 5 shows components of the delivery device applied to a bag.

### DETAILED DESCRIPTION OF PREFERRED EMBODIMENTS OF THE INVENTION

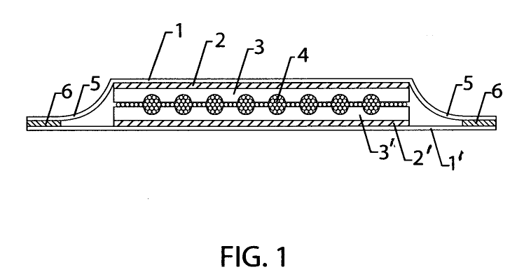

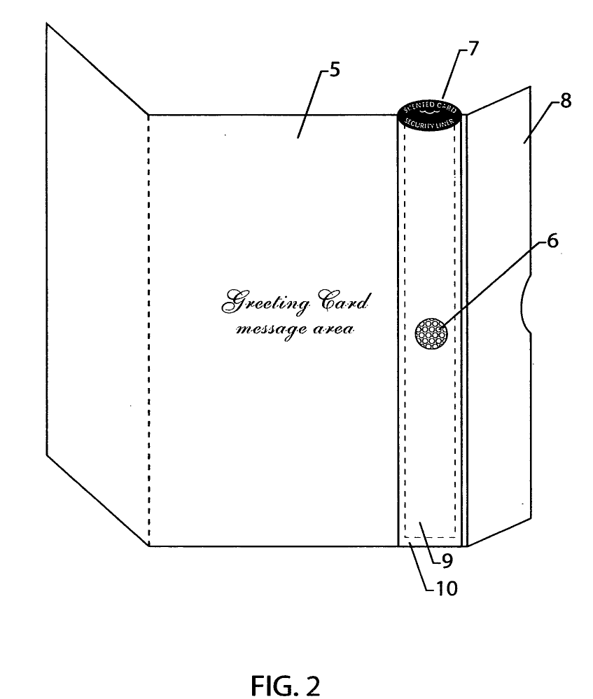

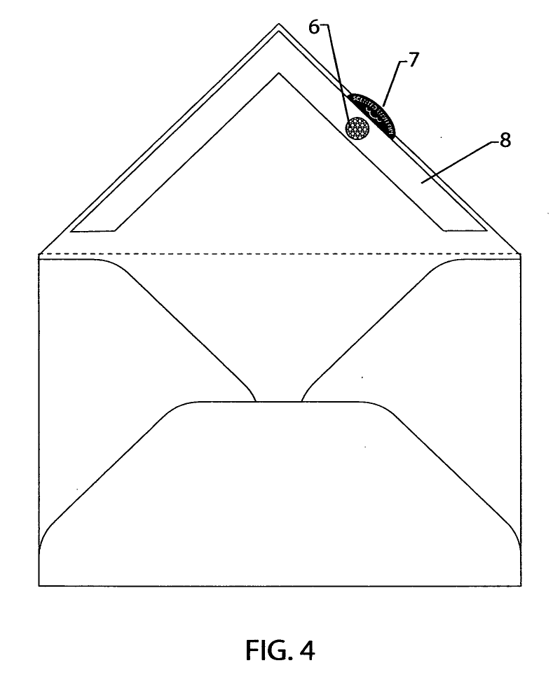

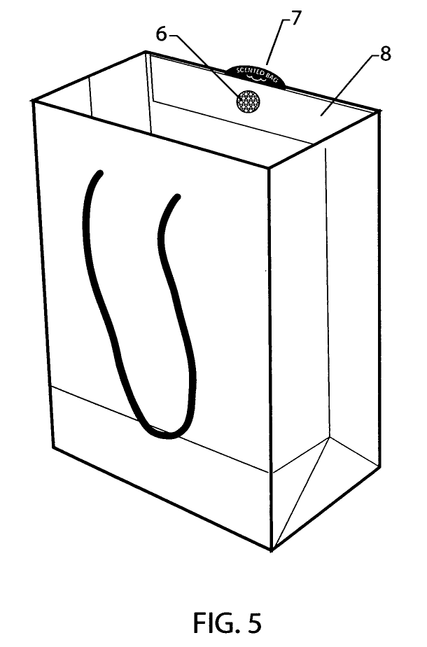

Referring now to FIG. 1, a scent delivery device employing the scent strip is illustrated, and is used on surfaces that face each other and separate from one another such as surfaces of greeting cards (FIG. 2), envelopes (FIG. 4) and bags (FIG. 5). The scent strip is comprised of two elastomeric layers 3 and 3′, preferably of plastic foam, that sandwich a microencapsulated scented slurry 4 which, when in an inactive state, remains inert. When the device is finally activated when the substrate layers 3 and 3′ are separated or deployed, the micro-encapsulated scented slurry breaks apart and releases the scented material into the surrounding environment, thereby enhancing the sensory impact and overall market value of the associated product. Elastomeric layers 3 and 3′ cushion the micro-capsules to deter wasteful and undesired premature release of scent during handling.

The consumer of the product, initially activates the scent strip by manually removing an adhesive backing layer, or security release liner 1, that exposes an adhesive surface 2. This process initially activates the scent strip, so when the accompanying product is later opened or deployed, the micro-encapsulated scented slurry layer breaks apart and the scent is deployed.

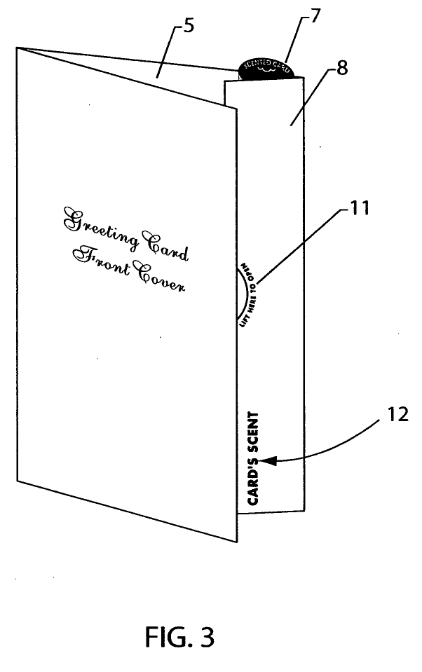

In FIGS. 2 and 3, a greeting card 5 has the delivery system of FIG. 1 affixed thereto by means of lower adhesive layer 2′ of FIG. 1. The adhesive layer 2′ is first exposed by removing liner 1′ before the affixing step. Market activation tab 7 is affixed to protective security release liner 9, corresponding to liner 1 of FIG. 1, to aid in peeling away protective liner 9 from the card. Tab 7 and/or liner 9 has indicia thereon indicating that the card has a desirable scent releasing capability.

After removal of the security release liner 9, by pulling on tab 7, the sender of the card folds scent activation panel 8 over the now exposed upper adhesive layer 2 of FIG. 1 to adhere it to the card via adhesive layer 2 shown in FIG. 1. The card is then mailed to the recipient who is directed to open the scent activation panel 8 by the note printed on the card at portion 11, shown in FIG. 3.

However, should the card sender forget to separate the security release liner from the card, tab 7 will extend over the edge of the card as shown in FIGS. 2 and 3 and as a result, the card cannot be inserted into the envelope that comes with the card. This functions to ensure that the card has become initially activated by the card recipient, enabling subsequent scent deployment, before its being sent to the card recipient.

Note that the upper security release liner 1 at portion 5 in FIG. 1, protects the edges of the scent strip, and in like manner liner 9 overlays the edge of the scent strip at 10 in FIG. 2, producing a beneficial protective edge seal, since liner 9 carries a weak non-permanent adhesive. This deters a customer from peeling away the liner in the store with her nail, prematurely exposing the upper adhesive 2 and/or breaking the micro-encapsulated scented flurry layer.

It is an additional feature of the invention that protective security release liner 9 has a small window 6 therein, enabling a prospective greeting card purchaser to scratch the scent strip and sniff a small amount of sampled scented material passing through the small window from the scent material containing substrate, and yet preventing undesirable release of substantial scent material within a store carrying the greeting cards. Such undesirable release of substantial scent material can be very offensive to persons in a store selling the greeting cards, and furthermore such premature release wastes the scent that the recipient of the cards should enjoy.

To summarize somewhat, market activation tab 7 serves three purposes: it differentiates the product in the retail environment as “Scented” thereby attracting additional attention from competing products, as the tab informs consumers that the product is scented, and can also indicate the particular scent carried by the card. Tab 7 also serves as a gripping point for the aforesaid initial activation enabling subsequent scent deployment. Furthermore, the tab actually prevents the consumer from accidentally giving or sending the product without initially activating it, as in the case with greeting cards, as the card will not fit into the envelope until the card has been initially activated by pulling on tab 7 to remove the adhesive liner 9 coupled to the tab. In the case of gift bags (FIG. 5), the tab 7 can fold over the bag opening, forcing the consumer to physically move the tab out of the way when loading the bag, and serves as a reminder to seal or initially activate the scent system by removing activating panel 8 by pulling on the tab. Sample window 6 previously described, can be employed as shown in FIGS. 4 and 5.

In FIG. 4, the envelope user pulls on tab 7 to peel away the security release liner 8 to expose the underlying adhesive layer that seals the envelope flap to the body of the envelope. Upon receipt of the sealed envelope, typically through the mail, the envelope recipient opens the envelope to cause the release of the scent through rupture of the micro-capsules.

The scent delivery device of FIG. 1 may be manufactured separate from the aforesaid products, thereby allowing existing manufacturing methods for the accompanying product to remain the same and even allow upgrading of existing products. This independent manufacturing process isolates the environmental issues associated with micro-encapsulation manufacturing. The lower protective film 1′ is removed upon the delivery device being affixed to the product by the action of lower adhesive layer 2′.

The highly compliant, elastomeric layers 3 and 3′, that are preferably plastic foam, serve several specific beneficial functions, such as:

(A) establishing a substantial thickness of the strip, thereby making the strip's surface level substantially higher than the rest of the accompanying product's surface, which facilitates a more complete adhesion of the scent strip to the accompanying product's surface during the aforesaid initial activation step. The higher the elastomeric layers 3 and 3′ the greater the yielding to product surface differentiation, such as uneven or embossed surfaces, frequently found in greeting cards and gift bags. This higher surface level also creates a more complete seal on the scent strip's exposed upper adhesive surface as its greater height level results in more sealing pressure applied directly on the scent strip area.

(B) the elastomeric substrate contains scent cavities 4. (FIG. 1), that can be small grooves embossed into the elastomeric material, that create channels that accommodate substantial volumes of scented material, which translates to great initial scent impact when the scent strip is first deployed, and allows for greater subsequent scent releases as the channels hold a reserve portion of the scented material for subsequent deployment for the recipient to enjoy. The cavities 4 could also constitute dimples as in the surface of a golf ball.

(C) The elastomeric substrate acts as a cushion and protects the micro-encapsulated bubbles from rupturing during manufacturing, shipping and handling. This cushioning effect will help preserve more micro-encapsulated scented oil thereby maximizing the scent release when deployed.

Elastomeric substrates for use in implementing the invention can be plastic foam products referred to in U.S. Pat. No. 4,791,143 issue to Tanaka et al., Dec. 13, 1988, incorporated by reference herein.

The FIG. 1 configuration of channels serve a unique function during deployment when the scent strip is pulled apart. The scent cavities such as channels are separated from one another by a connecting layer of micro-encapsulated slurry, substantially thinner than heights of the scent channels, and thus an adhesion variation break pattern is provided upon separation of these substrates during scent deployment. This break pattern is perceived as a distinctive beneficial tactile and auditory feedback during scent deployment.

D. The scent strip's security release liners 1 and 1′ in FIG. 1, peel off and expose the adhesive layers 2 and 2′ respectively. These liners protect the adhesive layers on both sides of the scent strip until they are needed, one liner is removed during the manufacturing/assembly process to adhere the Scent Strip in place on the product, and can thus be called the “Production Assembly Liner”. The other side is very similar, however, this liner is removed by the customer just prior to initial activation of the product and adheres to the products opposing panel, flap or bag panel to be opened, which effects final activation to deploy the scent. Thus this liner is called the “Customer Activation Liner”. These liners have several features and benefits, they are as follows:

(D) i. The “Customer Activation Liner” is combined with the “Marketing Activation Tab” allowing the consumer to identify the product as scented when in a display rack as the Tab extends above the top edge of the product.

ii. The “Customer Activation Liner”, the area below the Tab provides a large area to carry printing to instruct the consumer regarding the benefits and usage of the scented product.

iii. The “Customer Activation Liner” can be manufactured separately from the scent strip. This independent component provides wide flexibility in production techniques allowing the liner and tab to be die-cut into precise shapes and sizes thereby accommodating any size and shaped product.

iv. The “Scent Sample Area” is a scratch and sniff window 6 that is formed within the “Customer Activation Liner” allowing consumers to sample the cards scent in the retail marketplace. The Scent Sample Area is just big enough to offer a sample of the scent, but will not contaminate the retail environment.

v. “Edge Seals” offers an additional level of protection to the Scent Strip by having the “Customer Activation Line” 9—slightly overlapping the elastomeric plastic foam substrate material at border portions 10, as shown in FIG. 2, and sealing this overlap directly to the product with a weak non-permanent adhesive. This extra measure ensures that the Scent Strip's edges are not exposed and cannot be tampered with as they are completely covered by the sealed overlap.

While the invention has been described in connection with preferred embodiments, the description is not intended to limit the scope of the invention to the particular forms set forth, but on the contrary, it is intended to cover such alternatives, modifications, and equivalents as may be included within the spirit and scope of the invention as indicated by the language of the appended claims. For example, the term “cavities” includes channels, or other patterns of indentations in substrates 3 and 3′ in FIG. 1. For example the cavities can be similar to the dimples on the surface of a golf ball that may be produced by embossing.

What is claimed is:

1. A greeting card comprising:
(a) a scent strip mounted upon a portion of said greeting card having a scent material containing substrate including a slurry of micro-encapsulated scented material along with an adhesive layer;
(b) a protective security release liner overlying said adhesive layer;
(c) a scent activation panel coupled to said greeting card;
(d) and wherein a tab is affixed to said protective security release liner to aid in peeling said protective security release liner off of said greeting card while preventing said greeting card from being inserted into an envelope associated with said greeting card by virtue of said tab extending over an edge of said greeting card, unless the protective security release liner is separated from said greeting card, enabling subsequent scent release by a recipient of said card upon removal of said scent activation panel from said scent strip.

2. The greeting card of claim 1 wherein said greeting card has indicia thereon indicating that the greeting card has a scent releasing capability and can indicate a particular scent.

3. The greeting card of claim 1 wherein said scent material containing substrate is made of an elastomer.

4. The greeting card of claim 3 wherein said elastomer constitutes a plastic foam.

5. The scent delivery device of claim 1 wherein said substrate has a pattern of cavities therein containing micro-encapsulated scented material, said cavities being separated from one another by a connecting layer substantially thinner than heights of said scent channels.

6. The scent delivery device of claim 3 wherein said substrate has a pattern of cavities therein containing micro-encapsulated scented material, said cavities being separated from one another by a connecting layer substantially thinner than heights of said scent channels.

7. The scent delivery device of claim 4 wherein said substrate has a pattern of cavities therein containing micro-encapsulated scented material, said cavities being separated from one another by a connecting layer substantially thinner than heights of said scent channels.

8. A greeting card comprising:
(a) a scent strip mounted upon a portion of said greeting card having a scent material containing substrate including a slurry of micro-encapsulated scented material along with an adhesive layer;
(b) a protective security release liner overlying said adhesive layer;
(c) a scent activation panel coupled to said greeting card;
(d) and wherein said protective security release liner has a small window therein, enabling a prospective greeting card purchaser to scratch said scent strip and sniff a small amount of sampled scented material passing through the small window from said scent material containing substrate, and yet preventing undesirable release of substantial scent material within a store carrying the greeting cards.

9. The greeting card of claim 8 wherein said greeting card has indicia thereon indicating that the greeting card has a scent releasing capability and can indicate a particular scent to be released by the greeting card.

10. The greeting card of claim 8 wherein said scent material containing substrate is made of an elastomer.

11. The greeting card of claim 10 wherein said elastomer constitutes a plastic foam.

12. The scent delivery device of claim 8 wherein said substrate has a pattern of cavities therein containing micro-encapsulated scented material, said cavities being separated from one another by a connecting layer substantially thinner than heights of said scent channels.

13. The scent delivery device of claim 10 wherein said substrate has a pattern of cavities therein containing micro-encapsulated scented material, said cavities being separated from one another by a connecting layer substantially thinner than heights of said scent channels.

14. The scent delivery device of claim 11 wherein said substrate has a pattern of cavities therein containing micro-encapsulated scented material, said cavities being separated from one another by a connecting layer substantially thinner than heights of said scent channels.

15. The greeting card of claim 8 wherein a tab is affixed to said protective security release liner to aid in peeling said protective security release liner off of said greeting card while also preventing said greeting card from being inserted into an envelope associated with said greeting card, unless the protective security release liner is separated from said card, enabling subsequent scent release by a recipient of said greeting card upon separation of said overlying scent activation panel from said scent strip.

16. The greeting card of claim 10 wherein a tab is affixed to said protective security release liner to aid in peeling said protective security release liner off of said greeting card while also preventing said greeting card from being inserted into an envelope associated with said greeting card, unless the protective security release liner is separated from said card, enabling subsequent scent release by a recipient of said greeting card upon separation of said overlying scent activation panel from said scent strip.

17. A scent delivery device that can be affixed to various products comprising:
(a) an elastomeric substrate containing micro-encapsulated scented material;
(b) a first adhesive layer contacting a first surface portion of said elastomeric substrate;
(c) a second adhesive layer contacting a second surface portion of said elastomeric substrate opposite said first surface portion;
(d) a first protective security release liner positioned upon said first adhesive layer; and
(e) a second protective security release liner positioned upon said second adhesive layer.

18. The device of claim 17 wherein said elastomeric substrate constitutes a plastic foam.

19. The scent delivery device of claim 17 wherein said substrate has a pattern of cavities therein containing micro-encapsulated scented material, said cavities being separated from one another by a connecting layer of micro-encapsulated slurry substantially thinner than heights of said scent cavities.

20. The scent delivery device of claim 18 wherein said substrate has a pattern of cavities therein containing micro-encapsulated scented material, said cavities being separated from one another by a connecting layer of micro-encapsulated slurry substantially thinner than heights of said scent channels.

21. The scent delivery device of claim 17 wherein said protective security release liner has a small window therein, enabling a prospective greeting card purchaser to scratch said scent strip and sniff a small amount of sampled scented material passing through the small window from said elastomeric substrate, and yet preventing undesirable release of substantial scent material.

22. The scent delivery device of claim 18 wherein said protective security release liner has a small window therein, enabling a prospective greeting card purchaser to scratch said scent strip and sniff a small amount of sampled scented material passing through the small window from said elastomeric substrate, and yet preventing undesirable release of substantial scent material.

23. The scent delivery device of claim 19 wherein said protective security release liner has a small window therein, enabling a prospective greeting card purchaser to scratch said scent strip and sniff a small amount of sampled scented material passing through the small window from said elastomeric substrate, and yet preventing undesirable release of substantial scent material.

24. The scent delivery device of claim 20 wherein said protective security release liner has a small window therein, enabling a prospective greeting card purchaser to scratch said scent strip and sniff a small amount of sampled scented material passing through the small window from said elastomeric substrate, and yet preventing undesirable release of substantial scent material.

25. The greeting card of claim 17 wherein said greeting card has indicia thereon indicating that the greeting card has a scent releasing capability and can indicate a particular scent to be released by the greeting card.
